(19) United States
(12) Patent Application Publication
(10) Pub. No.: US 2016/0206306 A1
(43) Pub. Date: Jul. 21, 2016

(54) METHOD FOR FIXATION OF TISSUE AND DEVICE USED IN THE METHOD

(71) Applicant: BIORETEC OY, Tampere (FI)

(72) Inventors: Harri Heino, Tampere (FI); Joonas Mikkonen, Tampere (FI)

(21) Appl. No.: 14/600,913

(22) Filed: Jan. 20, 2015

(51) Int. Cl. A61B 17/04; A61F 2/08; A61B 17/88; A61B 17/16

(52) U.S. Cl. CPC A61B 17/0401; A61B 17/1615; A61F 2/0805; A61F 2/0811; A61B 17/8869; A61B 17/88; A61B 2017/0464; A61B 2017/0409; A61B 2017/044; A61F 2002/0876; A61B 2017/90

(57) ABSTRACT

A method for fixation of soft tissue to bone. A permanent fixing site of the tissue in the bone is selected. A temporary locking site of the tissue in the bone is selected. The temporary locking site is at a distance from the permanent fixing site. A fixing length of the tissue is selected that corresponds a tension state of the tissue. The tissue is temporarily fixed at the permanent fixing site. The tissue is locked at the temporary locking site in a position corresponding to the fixing length. The tissue is permanently fixed to the permanent fixing site. The locking of the tissue is released at the temporary locking site after permanently fixing the tissue. The locking of the tissue at the temporary locking site is carried out by a holder that has a lock to contact the bone, and a grip separate from the lock for gripping the tissue.

### FIELD OF THE INVENTION

The present invention relates to the fixation of tissue, especially fixation of tendon, ligament or soft tissue to bone.

### BACKGROUND OF THE INVENTION

In surgical re-attachment of soft tissue to bone, many methods and devices have been developed to anchor the soft tissue (tendon, ligament) to bone. The surgical operation where the soft tissue is attached takes place after a traumatic injury or in reconstructive surgery, and the tendon or ligament can be attached to the bone by various techniques. One typical way is to pass the tendon through the bone with sutures and fix it under tension with interference screws, as is shown for example in Clanton, T. and Perlman, M. “Interference Screw Fixation of Tendon Transfers in the Foot and Ankle”, Foot & Ankle Intl. 23(4):355-356, 2002. Drilling through the bone for making the transosseous tunnel for fixation is, however, traumatic for the tissue behind the bone and it weakens the bone. A technique for avoiding the transosseous tunnel includes the drilling of a blind hole or socket in the bone and fixing the tendon with the help of an interference screw, which is fastened to the socket by using a special driver. The procedure and the pertinent tools are disclosed in U.S. Pat. No. 6,544,281. In this procedure, the driver which is used for screwing the interference screw into the socket is also used for holding the sutures and applying sufficient tightness to them through a traction suture exiting the driver handle at the proximal end. For performing this function, the shaft of the driver must be cannulated from the proximal end all the way to the distal tip so that sutures can be held and controlled by a suture loop of the traction suture emerging from the cannulated shaft at the tip.

Thus, the tendon must be set to a proper tension before it is permanently attached to the bone. This requires that the tendon is positioned exactly with respect to the socket before the interference screw or corresponding fixing means is screwed down or attached in other way to the socket to immobilize the tendon. If this is done with the same tool that is used to fix the fixing means, the tool must be made complicated.

### SUMMARY OF THE INVENTION

It is the purpose of the invention to provide a method and tool system which allows the tensioning of the tendon independently of the fixing phase of the tendon during surgical operation to a human patient. The tendon is locked temporarily to the bone under selected tension or to a position corresponding to the selected tension by means of a holding device which is placed on the bone at a distance from the permanent fixing site, such as a socket. The holding device locks the tendon to the bone after the tendon has been guided to the fixing site and set to a proper tension at the fixing site. While the tendon is temporarily locked to the bone, the tendon can be attached permanently to the fixing site for example by screwing down an interference screw into a predrilled socket. The tendon may have sutures attached to its free end in a known manner, and these sutures can be used for guiding the tendon to the fixing site and setting it to the required tension.

The holding device is a gripper which may have a shaft with a tip for penetration into the bone as well as a spiked washer provided for rotation around the shaft but immovable along the length of the shaft. The shaft can be designed as a typical K-wire (Kirschner wire), a thin metal wire commonly used in surgery. Once the tendon is tensioned to the selected tension in a proper position with respect to the fixing site and the correct fixing length of the tendon is determined thereby, the tendon is ready for permanent fixation. The shaft of the holding device is screwed down to the bone so that it pierces the tendon, and the spikes of the washer grip the tendon and hold it in place with minimum damage to the tendon. Another embodiment for the gripper is a simple clamp which has first spikes for contact with the bone and second spikes for gripping the tendon.

The tendon can be guided to the fixing site and set to the suitable tension in the fixing site by means of a guiding tool which also serves as a temporary fixing member for the tendon at the fixing site when the tendon is locked to the bone by means of the holding device.

The guiding tool in the tool system may be a specially designed spoon which is used for guiding the tendon to the socket and for fixing the tendon in the socket in the correct fixing length before the tendon is temporarily locked to the bone. The spoon has a chute-shaped tip section which can accommodate the tendon and which can be used to guide and push the tendon to the bottom of the socket before placing the interference screw or corresponding fixing means to the socket.

The invention enables use of fixing means, such as a screw, and a fixing tool, such as a driver, which are simple in construction. The screw and the driver need not be cannulated to accommodate sutures for tensioning the tendon, but it is enough that they have engaging parts for non-rotative connection between the screw and the driver. The tip of the driver can be for example solid which increases the strength of the tool.

### BRIEF DESCRIPTION OF THE DRAWINGS

The invention will be explained in more detail with reference to the appended drawings. In the drawings

FIG. 1 shows a holding device to be used in the method in a side view,

FIG. 2 shows a detail of the holding device in cross-section,

FIG. 3 shows a guiding tool to be used in the method,

FIG. 4 illustrates the use of the holding device and the guiding tool during the method,

FIG. 5 is a general view of a holding device according to another embodiment,

FIG. 6 is a bottom plan view of the holding device of FIG. 5, and

FIG. 7 is an example of a drill bit that can be used for making the fixing site in the method.

### DETAILED DESCRIPTION OF THE EMBODIMENTS

The holding device for temporary locking of the tendon is a gripper which is designed to grip the tendon with its gripping means and hold it under the selected tension or in a correct position corresponding to the selected fixing length to which the tendon has been set at the fixing site. For the temporary locking of the tendon, the holding device is positioned at a selected site (temporary locking site) on the bone with its locking means.

In FIG. 1, the holding device 1 has a shaft 1c, around which is placed a washer 1b, which has spikes 1a pointing in the same direction as the pointed tip 1e of the shaft. The spikes 1a are distributed over the periphery of the washer. The washer 1b is rotatable around the shaft 1c, but its translational movement in the longitudinal direction of the shaft is prevented, and the washer and the spikes 1a are thus at a constant distance from the tip 1e. The movement further away from the tip is prevented by a stopper 1d which is attached to the shaft 1c. The shaft 1c acts like a K-wire (Kirschner wire), and it has threads in the front section between the washer 1b and the tip. The shaft can have dimensions and it can be made of materials commonly used in K-wires. Typical diameters of K-wire, applicable also in the holding device, are 0.7-2.0 mm.

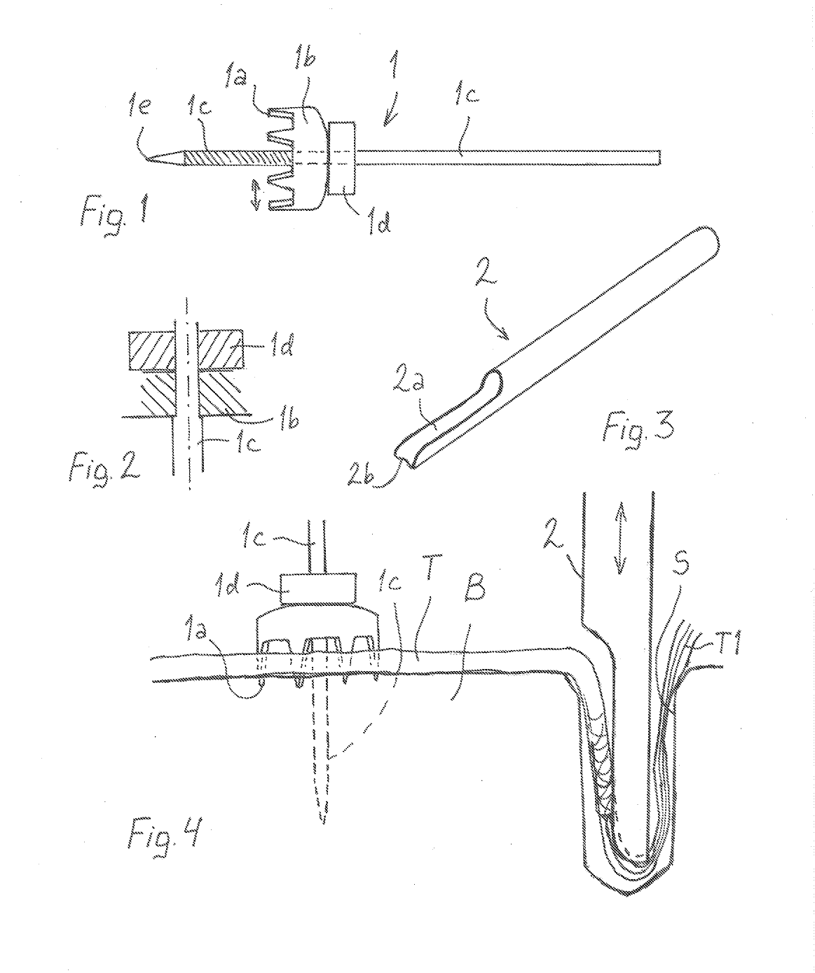

FIG. 2 shows the attachment of the washer 1b to the shaft 1c. The shaft has a shoulder which prevents the movement of the washer forward to the tip 1e and the movement rearwards is prevented by the stopper 1d. The shaft is received in the central hole of the washer with a sufficient clearance so that the washer can rotate around the shaft. There are other possibilities to manufacture the device and fix the washer 1b to the shaft so that the washer is rotatable at a predetermined distance from the tip 1e.

FIG. 3 shows a guiding tool 2 which can be used as a part of a tool system, for example in a surgical kit comprising any of the holding devices presented in this disclosure and possibly other surgical instruments. The guiding tool is a sort of spoon, whose tip section 2a is formed as a tongue and which has a shaft extending to the rear from the tip section 2a. The tool of FIG. 3 has a chute-shaped tip section 2a, which forms a groove for receiving the tendon during the manipulation. The distal end of the tip section is notched (notch 2b) to better guide the sutures attached to the tendon. The tool can be easily made of a tubular blank of suitable length, whose end is cut to the shape of chute. This manufacturing technique is preferable if the tool is made of metal.

FIG. 4 shows the position of the tools of the tool system during the surgical operation. A permanent fixing site on the bone B for the free end of the tendon T is selected, in case of FIG. 4 by making a blind-drilled hole or socket S in the bone B. The tendon T has sutures T1 attached to its end in a manner known as such. The tendon T is set to a right tension in the following way;

The tendon is placed to the permanent fixing site, in case of FIG. 4 to the socket S. The guiding tool 2 is used to guide the tendon T to the socket S with the help of the chute-shaped tip section 2a which accommodates the sutures T1 in the groove. The free end of the tendon T can be drawn gradually tighter as the tool is moved up and down the socket (as shown by double-headed arrow) and by pulling the sutures on the back side of the tool with the other hand in a short jerk every time as the tool is lifted. When the tendon T is in appropriate tightness after the last jerk, the tool is pushed down in the socket where it temporarily fixes the tendon T (the sutured end of the tendon) in the socket S in the selected fixing length where the tendon has the desired tension.

In this position of the tendon T, the holding device 1, which is the holding device of FIG. 1 described above, is applied on the bone to a selected provisional locking site where it keeps the tendon T in a position which corresponds to the selected fixing length and the selected tension of the tendon. The provisional locking is achieved by inserting the front section of the shaft 1c to the bone to a depth where the spikes 1a of the washer 1b come into gripping contact with the tendon T, and as shown by FIG. 4, even come into contact with the bone B. If the shaft 1c is inserted into the bone B by rotating it, the spikes 1a and the washer 1b remain stationary against rotation once the spikes 1a have entered into contact with the tendon T, and they grip deeper in the tendon as the shaft is advanced further into the bone.

The front section of the shaft 1c forms the locking means of the holding device and the spikes 1a form the gripping means of the holding device.

The shaft 1a can be driven to the bone by a drill for example, or manually.

The proximal portion of the shaft 1c (extending rearwards from the washer 1b) can also be used as a sort of handle which can be used for holding the bone in place during the operation This may be useful in cases where the holding device is attached to a small bone with high mobility, like e.g. patella.

After the tendon T is temporarily locked to the bone at the temporary locking site, which is at a distance from the permanent fixing site and between the opposite end (tissue attachment point) of the tendon and the permanent fixing site, the guiding tool 2 (which has served as temporary fixing member for temporarily fixing the tendon at the permanent fixing site) is removed from the socket and the permanent fixing means is placed in the socket. The permanent fixing means can be for example an interference screw, which fixes the tendon finally in the socket. Simple drivers can be used for screwing the interference screw down into the socket since no arrangements for attaching the sutures to the driver need to be made. The interference screws need not be cannulated either, or the cannulation at the tip of the screw (distal end) can be smaller in diameter than the tip of the driver. The tip of the driver can be solid with no cannulation. All other types of insertable permanent fixing means can also be used, e.g. plug-type means, in which case some other types of fixing tools may be used for inserting the plug. The fixing means is made preferably from bioabsorbable polymer with possible additives, such as bone growth promoting additives.

One advantageous alternative is to use bioabsorbable screws made by the Applicant and known under trademark “ActivaScrew”. These screws are characterized by contraction in the length of the screw and related expansion in the diameter of the screw under tissue conditions, that is, when implanted in a living body. The screws and suitable materials for them are described in published European Patent Application EP2127608.

After the tendon is permanently fixed in some suitable way to the permanent fixing site, the temporary locking means is removed. The holding device 1 of FIG. 1 can be pulled out of the bone B. The tendon runs now from its opposite end (tissue attachment point) to the permanent fixing site under proper tension.

It is enough that a correct fixing length of the tendon corresponding the desired tension in one position is selected for the fixation. This tension can be called a first tension state. The fixing length can then be locked by the temporary fixing means at the temporary locking site close to the permanent fixing site. This selected tension does not necessary exist at the time of the permanent fixation, because the permanent fixation of the tendon to the permanent fixing site may take place in another position where the tension is decreased or relieved (the second tension state). Thus, before the permanent fixation, the bone can be moved so that the position of the permanent fixing site with respect to the opposite end (tissue attachment point) of the tendon changes. However, because the length of the tendon is locked to correspond the correct tension (the first tension state) in one position, the tendon will have the correct length at the time of permanent fixation and will assume the correct tension once the bone moves back to the said position.

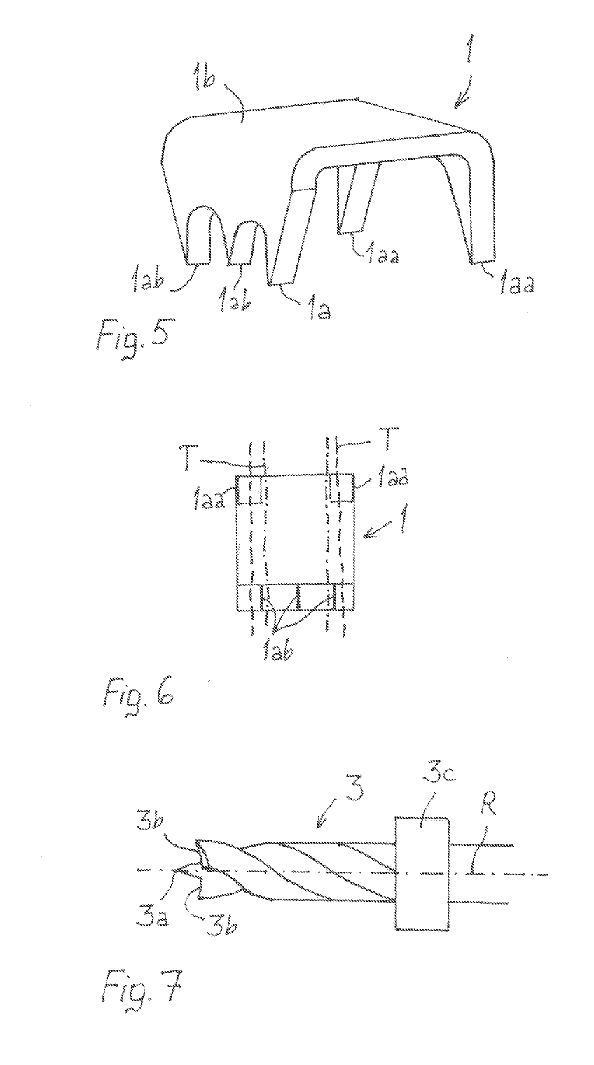

FIGS. 5 and 6 show another embodiment of the holding device. The holding device 1 is a simple clamp, a sort of thumb tack or push pin where several spikes protrude from the body 1b of the device in one direction. The holding device has first spikes 1aa for contact with the bone (locking means) and at a distance from the first spikes, second spikes 1ab for gripping the tendon (gripping means).

As is best shown in FIG. 6, the first spikes 1aa at one end of the clamp body are laterally offset outwards with respect to the second spikes 1ab at the other end. Thus, the first spikes 1aa remain outside the longitudinal zone defined by the outermost spikes of the second spikes 1ab. The tendon T illustrated with broken line in FIG. 6 will be gripped by the second spikes 1ab only while the first spikes 1aa are in locking contact with the bone and the tendon runs between the first spikes 1aa. Its is also possible that that the mutual widthwise dimensions of the clamp are such that the tendon also runs between the outermost spikes of the second spikes 1ab and is gripped only by the spike or spikes between these outermost spikes, as is illustrated by dash-and-dot line in FIG. 6. The number of the second spikes 1ab is at least two, preferably more than two, most preferably three or four.

Among the second spikes 1ab, spikes have different lengths, that is, penetration depths. In FIG. 5, the outermost spikes extend longer down than the middle spike, that is, if the clamp is placed on an even surface, there will be a space between the middle spike and the surface while the longer spikes touch the surface. If the tendon runs between the outermost spikes of the second spikes 1ab, the longer outermost spikes have an anchoring function to the bone (like the first spikes 1aa), while the shorter middle spike/middle spikes have the gripping function to the tendon.

The spike tips of the device of FIGS. 5 and 6 are sharp edges extending in the longitudinal direction of the spike body (which coincides with the direction of the tensioned tendon). This can be seen eg. in FIG. 6. By this shape, sufficient contact area but minimal damage to the penetrated tendon is achieved.

The clamp can be made of a plate like blank where the spike forms are cut at the opposite ends, where after the blank is bent at its both ends to the same side so that the body 1b is formed of the unbent middle portion. The clamp can be made also by casting to the same shape. Finishing operations to the material of the clamp may include sharpening the tips of the spikes. Metal is preferable material for the clamp.

The holding device 1 of FIGS. 5 and 6 must be held against the bone by one hand to cause the temporary locking, while the holding device of FIG. 1, more securely fastened to the bone, leaves both hands free.

The holding devices are preferably made of a surgical metal, such as stainless steel or other metal used for making surgical instruments. The guiding tool can be made of metal or plastic.

Finally, in FIG. 7, a drill bit 3 which is used for drilling the socket S in the bone is shown. The drill bit can be attached to a conventional surgical drilling tool which brings the drill bit into rotation around its rotational axis R. Contrary to the conventional drill bits, this drill bit 3 has a sharp tip 3a coinciding with said rotational axis, and at a distance backwards from the tip, a pair of front cutting edges 3b extending sideways from the rotational axis R of the drill bit and having a larger radial extension than the tip. Said cutting edges 3b are at 180° distance from each other around the rotational axis. The drill bit has a stopper 3c placed at a predetermined distance from the tip 3a to limit the depth of the socket. The sharp tip 3a can be used to exactly position the drill bit 3 to the place where the socket is to be formed when the permanent fixing site is selected, by pressing the tip down to the bone, and subsequently for guiding the front cutting edge 3b behind the tip 3a to the bone. The drill bit is also made of surgical grade metal, such as stainless steel.

All tools presented herein (holding devices, guiding tools, drill bits) are preferably supplied for surgical use in closed sterilized packages, but they can be sterilizable reusable instruments as well. Some or all of them can be included in a surgical kit.

The tendon has been mentioned as the example of soft tissue to be fixed. The tendon in this context shall mean tendon together with any sutures attached to its end. Further, what is said above about tendons applies likewise to ligaments. Further, the invention also covers allografts and prosthetic parts to be fixed to the bone, which will have the functions of tendons or ligaments.

The method can be used in any surgical operation performed on a human body, provided that there is enough space in the area of operation. The method and tools can be used for example in achilles tendon fixation and MPFL-fixation (medial patellofemoral ligament reconstruction/repair), to mention a few examples. The invention is not limited to human surgery, but it can be used also in veterinary surgery.

The invention is not limited to the above embodiments, but it is defined by the appended claims.

What is claimed is:

1. A method for fixation of soft tissue to bone during surgical operation, comprising:
selecting a permanent fixing site of the soft tissue in the bone;
selecting a temporary locking site of the soft tissue in the bone, the temporary locking site being at a distance from the permanent fixing site;
selecting a fixing length of the soft tissue that corresponds to a tension state of the soft tissue;
locking the soft tissue at the temporary locking site in a position corresponding to said fixing length;
fixing the soft tissue permanently to the permanent fixing site, and
after permanently fixing the soft tissue, releasing the locking of the soft tissue at the temporary locking site.

2. The method according to claim 1, further comprising:
after selecting said fixing length, temporarily fixing the soft tissue at the permanent fixing site.

3. The method according to claim 1, wherein the soft tissue is a tendon or ligament.

4. The method according to claim 1, wherein selecting a permanent fixing site comprises drilling a blind hole or socket at the permanent fixing site.

5. The method according to claim 1, wherein locking the soft tissue under tension comprises applying a holding device to the bone with a lock of the holding device in contact with the bone and with a grip of the holding device in gripping contact with the soft tissue.

6. A holding device, comprising:
a lock configured to contact with bone, and
a grip configured to grip soft tissue, wherein the grip is separate from the lock.

7. The holding device according to claim 6, further comprising:
a shaft having a front section,
a washer attached to the shaft rotatable around the shaft and immovable along the shaft,
spikes pointing in a direction of the front section of the shaft, wherein the spikes are in the washer,
wherein the front section of the shaft forms the lock of the holding device, and the spikes of the washer form the grip of the holding device.

8. The holding device according to claim 7, wherein the front section of the shaft ends in a pointed tip.

9. The holding device according to claim 7, wherein the front section of the shaft is threaded to facilitate the penetration of the holding device to the bone by rotating the shaft.

10. The holding device according to claim 8, wherein the front section of the shaft is threaded to facilitate the penetration of the holding device to the bone by rotating the shaft.

11. The holding device according to claim 6, further comprising:
a body,
first spikes for contact with the bone, wherein the first spikes protrude from the body, and
second spikes for gripping the soft tissue, wherein the second spikes protrude from the body in a same direction as the first spikes and at a distance from the first spikes, wherein the first spikes form the lock of the holding device and the second spikes form the grip of the holding device.

12. A surgical tool system, comprising:
a holder comprising a lock configured to contact with bone;
a grip configured to grip soft tissue, wherein the grip is separate from the lock;
a guide configured to manipulate the soft tissue and guiding the soft tissue to a permanent fixing site, the guide having a tip section in the form of a tongue and a shaft extending to a rear from the tip section.

13. The surgical tool system according to claim 12, wherein the holder comprises
a shaft having a front section,
a washer attached to the shaft rotatable around the shaft and immovable along the shaft, and
spikes pointing in the direction of a front section of the shaft, wherein the spikes are in the washer,
wherein the front section of the shaft forms the lock of the holding device, and wherein the spikes of the washer form the grip of the holding device.

14. The surgical tool system according to claim 12, wherein the holder comprises
a body,
first spikes for contact with the bone, wherein the first spikes protrude from the body,
second spikes for gripping the soft tissue, wherein the second spikes protrude from the body in a same direction as the first spikes and at a distance from the first spikes,
wherein the first spikes form the lock of the holding device, and wherein the second spikes form the grip of the holding device.

15. A surgical tool system, comprising:
a holder comprising a lock for contact with bone;
a grip for gripping soft tissue, wherein the grip is separate from the lock,
a drill bit having a rotational axis, a sharp tip coinciding with said rotational axis, and a pair of front cutting edges extending sideways from the rotational axis and having a larger radial extension than the tip, wherein the front cutting edges are at a distance backwards from said tip.

16. The surgical tool system according to claim 15, wherein the holding device comprises
a shaft having a front section,
a washer attached to the shaft rotatable around the shaft and immovable along the shaft, and
spikes pointing in the direction of the front section of the shaft, wherein the spikes are in the washer,
wherein the front section of the shaft forms the lock of the holding device, and wherein the spikes of the washer form the grip of the holding device.

17. The surgical tool system according to claim 15, wherein the holding device comprises
a body,
first spikes for contact with the bone, wherein the first spikes protrude from the body,
second spikes for gripping the soft tissue, wherein the second spikes protrude from the body in the same direction as the first spikes and at a distance from the first spikes,
wherein the first spikes form the lock of the holding device, and wherein the second spikes form the grip of the holding device.

18. The surgical tool system according to claim 15, further comprising:
a guide configured to manipulate the soft tissue and guide the soft tissue to the permanent fixing site, wherein the guiding tool has a tip section in the form of a tongue and a shaft extending to a rear from the tip section.
